(19) United States
(12) Patent Application Publication
(10) Pub. No.: US 2026/0357333 A1
(43) Pub. Date: Nov. 12, 2026

(54) Method and Device for Preamplifying a Nucleic Acid Mixture

(71) Applicant: Robert Bosch GmbH, Stuttgart (DE)

(72) Inventors: Franz Laermer, Weil Der Stadt (DE); Christian Grumaz, Waiblingen (DE); Sebastian Pilsl, Stuttgart (DE)

(21) Appl. No.: 19/121,678

(22) PCT Filed: Sep. 29, 2022
(86) PCT No.: PCT/EP2023/076999

§ 371 Date: Apr. 16, 2025

(30) Foreign Application Priority Data
Oct. 19, 2022 (DE) 10 2022 211 087.0

(51) Int. Cl. C12Q 1/6853; B01L 3/00; C12Q 1/48; C12Q 1/6806; C12Q 1/6827; C12Q 1/6876

(52) U.S. Cl. CPC C12Q 1/6853; B01L 3/502715; C12Q 1/48; C12Q 1/6806; C12Q 1/6827; C12Q 1/6876; C12Y 207/07007; C12Y 207/07049; C12Q 2600/156

(57) ABSTRACT

A method for preamplifying a nucleic acid mixture includes denaturing the nucleic acid mixture in order to obtain single-stranded nucleic acids, hybridizing the single-stranded nucleic acids with chemically modified primers, and isothermally preamplifying the hybridized nucleic acids by way of an SD polymerase. A microfluidic device includes an element for receiving a patient sample, an element for preamplifying a nucleic acid mixture in the patient sample, and an element for detecting nucleic acid biomarkers in the preamplified nucleic acid mixture. The device is designed so as to carry out the preamplification by way of the method.

The present invention relates to a method for preamplifying a nucleic acid mixture. Furthermore, the present invention relates to a microfluidic device configured so as to carry out a preamplification by means of the method.

### PRIOR ART

In molecular diagnostics, diseases can be detected based on nucleic acids that function as a biomarker. The nucleic acids may be DNA or RNA. In order to detect as many pathogens as possible simultaneously based on such biodevelopments, probe-based methods or sequencing can be used. However, in the case of low sample input material, preamplification must occur upstream of these methods, for example of the relevant bio-markers or of the overall mixture. However, a simultaneous preamplification of DNA and RNA is not possible in one reaction.

Preamplification may be carried out by thermophilic enzymes requiring cyclic change in temperature. This leads to an increased reaction duration, such that these preamplification reactions are not well suited for use in point-of-care. Isothermal amplification reactions are possible by means of mesophilic enzymes. However, these are less procedural than thermophilic enzymes, so that their use also leads to a very long reaction duration.

### DISCLOSURE OF THE INVENTION

The method for preamplifying a nucleic acid mixture, which is in particular contained in a patient sample, comprises denaturing the nucleic acid mixture in order to obtain single strands of the nucleic acids. The single strands of nucleic acids are subsequently hybridized with chemically modified primers. The hybridized nucleic acids thus obtained are subjected to isothermal preamplification by means of SD polymerase (strand displacement). The SD polymerase is a DNA polymerase suitable for isothermal amplification reactions and is already known for high strand displacement activity in amplification reactions. The method is based on the finding that SD polymerases are also suitable for preamplification reactions. The use of SD polymerase has several advantages. It allows isothermal preamplification without exhibiting the low processivity of mesophilic enzymes. In addition, some SD polymerases tolerate high temperatures during isothermal preamplification, which increase the reaction rate to such an extent that a significant reduction in reaction duration is possible. Finally, some SD polymerases may use RNA as a template for amplification in addition to DNA. The method thus exhibits only a small or no adjustment effort for different pathogens or biomarkers to be detected, because it can amplify DNA and RNA universally regardless of the application.

The nucleic acid mixture may be dissolved from a solid phase, for example silicon dioxide, with water or an elution buffer, in particular prior to denaturation. The denaturing is preferably carried out by heating the nucleic acid mixture dissolved in water, wherein the heating is preferably carried out to a temperature in the range of 75° C. to 98° C., and particularly preferably to a temperature in the range of 80° C. to 95° C. Preferably, the heating is carried out for a period of more than 10 seconds, particularly preferably for a period of time in the range of 30 seconds to 120 seconds. This reliably dissolves secondary and tertiary structures of the nucleic acids.

Preferably, the chemically modified primers for hybridization of the single-stranded nucleic acids may already be brought together with the nucleic acid mixture prior to denaturation, particularly preferably by being dissolved in the water or elution buffer. They are insensitive to the conditions of denaturation. Hybridization then preferably occurs at a temperature in the range of 4° C. to 50° C. for a period of time, preferably greater than 10 seconds. Under these conditions, the primers may attach to the single-stranded nucleic acids. The temperature required for the hybridization is achieved in particular by cooling a solution of the single-stranded nucleic acids obtained by denaturing.

To prepare the isothermal preamplification, the solution of the hybridized nucleic acids is mixed with the SD polymerase. Furthermore, in particular, deoxynucleoside triphosphates (dNTP) and buffer constituents may be added. The deoxynucleoside triphosphates serve as precursor building blocks of nucleic acids in the preamplification. The buffer constituents serve to adjust an optimal pH for preamplification. Furthermore, it is preferred that single-stranded binding proteins (SSBs) are added. The single-stranded binding proteins are proteins that bind to single-stranded DNA in order to facilitate the isothermal preamplification by supporting the SD activity of polymerase.

The isothermal preamplification is preferably only started when a homogeneous reaction mixture has formed from the solution of the hybridized nucleic acids and all further added reagents. The isothermal preamplification is preferably carried out at a constant temperature with a value in the range of 40° C. to 72° C. A preferred period of preamplification is in the range of 10 minutes to 120 minutes. The higher the temperature in the preamplification, the faster it will take place and the shorter the preamplification period can usually be advantageously chosen. Compared to preamplifications with mesophilic enzymes at 40° C. or less, this can achieve a significant reduction of the reaction time. The reaction is significantly accelerated, in particular at a temperature in the range of 45° C. to 65° C. The method also has the advantage that it can be used for preamplification of RNA even at such high temperatures, while other methods allow preamplification of RNA only at lower temperatures.

The primers are preferably primers having at least one modification selected from the group consisting of LNA (Locked Nucleic Acid), MGB (Minor Groove Binder), C-5 Propynyl-Deoxycytidine, C-5 Propynyl-Desoxyuridine, Aminoethyl-Phenoxazin Trisoxycytidine, 5-Methyl-Desoxycytidine, 2-Amino-Desoxyadenosine, Trimethoxystilbene, Pyrene, und Spermine. ZNA primers (Zip Nucleic Acids) are particularly preferred. These are spermine-modified primers. These have the advantage that they form particularly temperature-stable hybridized nucleic acids with single-stranded nucleic acids and that higher hybridization temperatures can be used without the need for sequence extension of the primers. They also reduce the self-hybridization of the primers if the primers are loaded with spermine. This is particularly advantageous when the primers used are short and the isothermal preamplification is carried out at temperatures of at least 45° C.

In a preferred embodiment of the method, the primers are modified random sequence primers (Random Primer). These primers, which in particular have a defined chain length, which is preferably between 6 and 15 bases, and consist of a purely random sequence, have the advantage that they are highly likely to cover all areas of the nucleic acid template to be amplified. They allow for an isothermal preamplification to be carried out as uMDA (universal Multiple Displacement Amplification).

In a further embodiment of the method, it is preferred that, in addition to the modified random sequence primers, specific sequence primers are used in order to hybridize the single-stranded nucleic acids. By the additional use of the specific primers, an ssMDA (semi-specific multiple displacement amplification) is enabled. These specific sequence primers can also carry modifications that increase the processivity of SD polymerase. In particular, these are spermine groups. Particularly preferred are specific sequence ZNA primers that increase the processivity of SD polymerase and have a repelling effect on each other during respective loading. This modification favors that there would be no unwanted interactions between the specific sequence primers. Furthermore, a high number of different specific sequence primers could be used as a result.

Specific sequence primers are understood to mean primers that not only have a defined chain length but also a defined sequence. This sequence may in particular be chosen in order to bind to certain loci on the nucleic acids, for example flanking one or more targets, thus favoring the accumulation of a target or any number relative to the background. Alternatively or additionally, the sequence may be selected in order to bind to sequences of the nucleic acids for which the SD polymerase has a lower processivity compared to other sequences of the nucleic acids, for example due to difficult sequences with high GC content. By amplifying the preamplification of the otherwise insufficiently amplified sequences, not only a universal but also a more uniform preamplification is enabled.

The specific sequence primers preferably have at least one selection marker, for example biotin. This selection marker is advantageous when the primers are specific to a target and are not only used in order to increase uniformity. In this case, it is possible to clean up or enrich the amplification products of the specific primers by means of their selection markers.

It is preferred for both modified random sequence primers and specific sequence primers that the primers have a single-stranded break interface. This single-stranded break interface provides an additional amplification start point for SD polymerase upon generation of the single-stranded break through a suitably added endonuclease (Nicking Enzyme). As a result, the preamplification at these locations may be continued without further primer hybridizations, which increases processivity.

During the preamplification, an endonuclease is added to the SD polymerase. The endonuclease is preferably selected from the group consisting of Nt.AlwI, Nb.BbvCI, Nt.BbvCI, Nb.Bsml, Nt.BsmAI, Nt.BspQI, Nb.BsrDI, Nb.BssSI, Nt.BstNBI, Nb.BtsI, and Nt.CviPII.

For example, the SD polymerase may be an enzyme mutant of the DNA polymerase of the Bacillus subtilis phage Phi29, in particular EquiPhi29, or the Thermococcus litoralis, in particular Vent (exo-). The SD polymerase vent (exo-) has the advantage that it is stable and processive up to temperatures of 100° C. Therefore, it may already be added to the nucleic acid mixture prior to denaturation without being damaged by the high denaturation temperature. However, these SD polymerases do not have RNA processivity.

Preferably, the nucleic acid mixture is a mixture containing both DNA and RNA, so that the possibility of universal preamplification reaction as provided by the method is exploited. Here, the SD polymerase is preferably an enzyme mutant of the DNA polymerase of the Bacillus stearothermophilus.In addition to their processivity towards DNA, these SD polymerases also exhibit processivity towards RNA. Particularly preferred enzyme mutants of the DNA polymerase of the Bacillus stearothermophilus are selected from the group consisting of Bst, Bst 2.0 and Bst 3.0. Among these enzyme mutants, Bst 3.0 has the highest RNA processivity, which is why Bst 3.0 is particularly preferred.

In the event that SD polymerases having 3′→5′ exonuclease activity are used, the primers may be modified with 3′-terminal phosphorothioate groups (PTO) and are thus protected.

Although the SD polymerase is capable of preamplifying RNA in addition to DNA, it is preferred that at least one reverse transcriptase is additionally used in the isothermal preamplification. In particular, this is RTx or SSIV. In principle, however, all reverse transcriptases that process under similar reaction conditions are conceivable, as is the SD polymerase used. This specifically supports the preamplification of RNA, so that the required preamplification period can be further reduced. If an SD polymerase that does not have RNA processivity is used, then the use of reverse transcriptase is necessary to process RNA in addition to DNA.

Because the method allows for isothermal preamplification in only a single reaction chamber in which the denaturation, the hybridization, and the isothermal preamplification are carried out, it is easily carried out in microfluidic devices or lab-on-chip systems, which enables automated analysis at the point of care without additional equipment. A microfluidic device comprising an element for receiving a patient sample is provided for this purpose. A further element may optionally be used in order to purify the nucleic acid mixture from the patient sample and to provide the preamplification in ultra-pure form. A further element for preamplifying a nucleic acid mixture in the patient sample is configured so as to carry out the preamplification by means of the method. Reagents for preamplification may preferably be positioned upstream in this element, particularly when provided in dry form as a bead. In so doing, the preamplification can take place directly with the patient sample without any preceding purification element or with the purified nucleic acid mixture. Finally, a nucleic acid biomarker detection element is provided in the preamplified nucleic acid mixture. There, for example, by adding specific primers and probes, target detection can be carried out via isothermal amplification. However, dilution and division of the preamplified nucleic acids into individual cavities can also be carried out in order to be able to detect a plurality of targets simultaneously and individually. Assay methods by means of qPCR, CRISPR/Cas, NGS, or microarrays may also be used for target detection.

### BRIEF DESCRIPTION OF THE DRAWINGS

Exemplary embodiments of the invention are illustrated in the drawings and explained in more detail in the following description.

FIG. 1 schematically shows a microfluidic device according to one exemplary embodiment of the invention.

FIG. 2 schematically shows the flow of a method according to one exemplary embodiment of the invention.

FIG. 3 schematically shows the flow of a method according to a different exemplary embodiment of the invention.

FIG. 4 schematically shows the flow of a method according to yet another exemplary embodiment of the invention.

FIG. 5 schematically shows the flow of a method according to yet another exemplary embodiment of the invention.

FIG. 6 schematically shows a microfluidic device according to a different exemplary embodiment of the invention.

FIG. 7 schematically shows the flow of a method according to yet another exemplary embodiment of the invention.

FIG. 8 schematically shows the flow of a method according to yet another exemplary embodiment of the invention.

### EXEMPLARY EMBODIMENTS OF THE INVENTION

In one exemplary embodiment of the invention, a microfluidic device is provided, which is designed for example as a disposable cartridge for an analysis device. Such cartridges comprise a fluidic layer, a pneumatic layer, and an elastomeric membrane arranged between the fluidic layer and the pneumatic layer. The fluidic layer and the pneumatic layer each comprise a substrate consisting of, for example, polycarbonate. For example, the elastomeric membrane is made of thermoplastic polyurethane. Channels and chambers are provided in the fluidic layer for conveying reaction liquids and reaction mixtures during the molecular diagnostics. Furthermore, the fluidic layer is provided with chemicals upstream for carrying out chemical reactions. The pneumatic layer comprises channels leading up to the elastomeric membrane and having openings on an outer side of the pneumatic layer. By means of these openings, the channels can be connected to a pneumatic manifold of the analysis device. When a positive pressure is generated by means of the pneumatic manifold in one of the individually controllable channels of the pneumatic layer, the elastomeric membrane is deflected into the fluidic layer. If, on the other hand, a negative pressure is generated in the channel, then the elastomeric membrane is deflected into the pneumatic layer. In this way, it is possible to manipulate fluid flows in the fluidic layer.

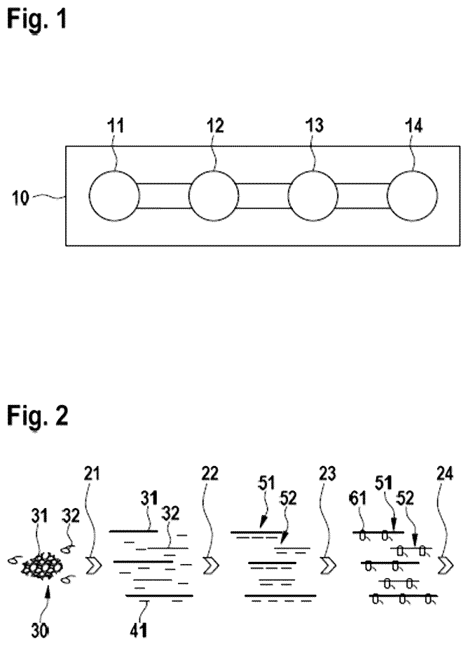

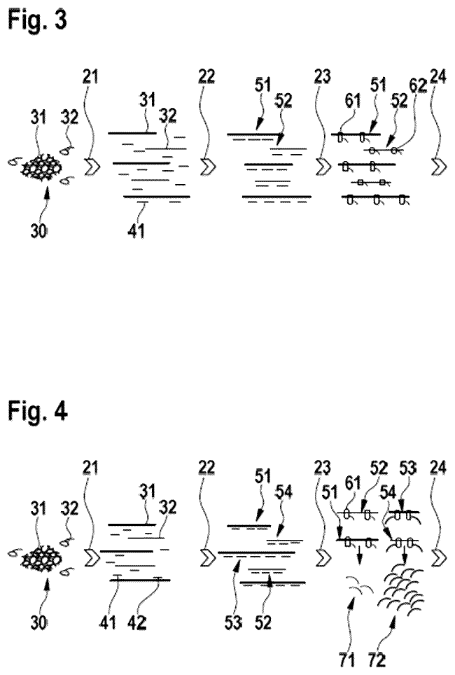

The illustration in FIG. 1 shows elements of the fluidic layer of the microfluidic device 10. A first element 11 is configured for receiving a patient sample. For this purpose, it comprises an opening on the outer side of the fluidic layer, through which the patient sample can be introduced and absorbed on silica gel in the first element 11. This first element 11 is fluidically connected to a second element 12 for cleaning the patient sample. The second element 12 is fluidically connected to a third element 13 for preamplification of a nucleic acid mixture contained in the patient sample. The third element 13 is fluidically connected to a fourth element 14 for purification of the preamplification product. This fourth element 14 is fluidically connected to a fifth element 15 for the detection of nucleic acid biomarkers in the purified preamplified nucleic acid mixture.

FIG. 2 shows method steps 21-24, which take place in a microfluidic device 10 in a first exemplary embodiment of the method according to the invention. As nucleic acids, the nucleic acid mixture 30 contains DNA 31 and RNA 32, which have secondary and tertiary structures. This nucleic acid mixture 30 is eluted in the first element 11 by means of an aqueous solution of ZNA primers and is pumped into the second element 12. There, denaturing of the nucleic acid mixture 30 is carried out as the first method step 21 by heating the solution to 95° C. for one minute. Thus, single-stranded DNA 31 and single-stranded RNA 32 is obtained, which is present in the solution with ZNA primers 41. For example, ZNA primers 41 are modified primers with three spermine groups on an N9-Oligo (random sequencing of 9 bases) (N9-ZNA3). The next method step 22 is to hybridize the single-stranded DNA 31 and the single-stranded RNA 32 with the ZNA primers 41. For this purpose, the solution is cooled to a temperature of 40° C., for example, and kept at this temperature for one minute. This yields primer-hybridized DNA 51 and primer-hybridized RNA 52. In the next method step 23, a preamplification of the hybridized DNA 51 and the hybridized RNA 52 is carried out. For this purpose, a further reaction solution is initially added to the solution from a chamber not shown in FIG. 1 and homogeneously mixed with the solution of hybridized DNA 51 and hybridized RNA 52. For example, the further reaction solution contains an SD polymerase 61 called Bst 3.0, as well as dNTPs, single-stranded binding proteins (SSBs), and a buffer. Then, the homogeneous reaction mixture is heated to a temperature of, for example, 50° C. for 30 minutes. Here, the SD polymerase 61 performs preamplification of the sequences hybridized by ZNA primers 41 on both hybridized DNA 51 and hybridized RNA 52 by means of strand displacement. Subsequently, a detection reaction 24 occurs. For this purpose, the reaction mixture is pumped into the third element 13. There, for example, it is distributed over a plurality of cavities of a microarray and is subjected to a qPCR reaction as the detection method. In so doing, the third element 13 comprises a window on its side facing away from the elastomeric membrane, which faces a sensor of the analysis device. This allows for an analysis of the reactions occurring in the cavities of the microarray by means of fluorescence spectroscopy.

In a second exemplary embodiment of the method according to the invention, it is provided that the reaction solution mixed in the second element 12 with the mixture of hybridized DNA 51 and hybridized RNA 52 also contains a reverse transcriptase 62, in addition to the constituents provided in the first exemplary embodiment. This is, for example, RTx. In the step of isothermal preamplification 23, a preamplification of the hybridized DNA 51 is carried out by the SD polymerase 61, as in the first embodiment. While the SD polymerase 61, as in the first exemplary embodiment, also preamplifies individual hybridized RNA strands 52, the preamplification of the hybridized RNA strands 52 in the second exemplary embodiment occurs after the reverse transcription to cDNA by virtue of the reverse transcriptase 62. The further method steps of the second exemplary embodiment of the method correspond to the first exemplary embodiment.

While in the first two exemplary embodiments, the preamplification 23 takes place as uMDA, the method according to the invention also allows for an ssMDA to be used in further exemplary embodiments:

In a third exemplary embodiment of the method according to the invention shown in FIG. 4, the elution solution introduced into the first element 11 also contains modified specific sequence primers 42 in addition to the modified ZNA random sequence primers 41. The modified specific sequence primers 42 have sequences that allow for hybridization of sequences of DNA 31 and RNA 32, for which SD polymerase 61 has only low processivity. For example, while the SD polymerase amplifies most sequences of DNA 31 and RNA 32 a thousand times by means of the modified ZNA random sequence primer 41, these nucleic acids may also have sequences that are amplified only twenty times. It is then contemplated that the modified ZNA specific sequence primers 42 are primers with sequences specific to these weakly amplifiable DNA sequences and RNA sequences. During the hybridization 22, as in the first two exemplary embodiments, hybridized DNA strands 51 that merely carry modified ZNA random sequence primers 41 and hybridized RNA strands 52 that only carry ZNA random sequence primers 41 are formed, on the one hand. In addition, hybridized DNA strands 53 are also formed that carry modified ZNA random sequence primers 41 and modified specific sequence primers 42. In addition, hybridized RNA strands 54 are formed that carry modified ZNA random sequence primers 41 and modified specific sequence primers 42. During the preamplification 23, SD polymerase 61 forms the same preamplification products 71 as can also be obtained in the first two exemplary embodiments of the method according to the invention from the hybridized DNA strands 51 and RNA strands 52, which only carry ZNA random sequence primers 41. In addition, by preamplifying the hybridized DNA strands 53 and RNA strands 54, which carry modified ZNA random sequence primers 41 and modified specific sequence primers 42, SD polymerase 61 forms a second amplification product 72, which has an increased proportion of the difficult-to-amplify sequence, on the sequences that have been attached to modified ZNA specific sequence primers 42. Otherwise, the flow of the method according to the third exemplary embodiment of the invention corresponds to the uMDA according to the first exemplary embodiment of the invention.

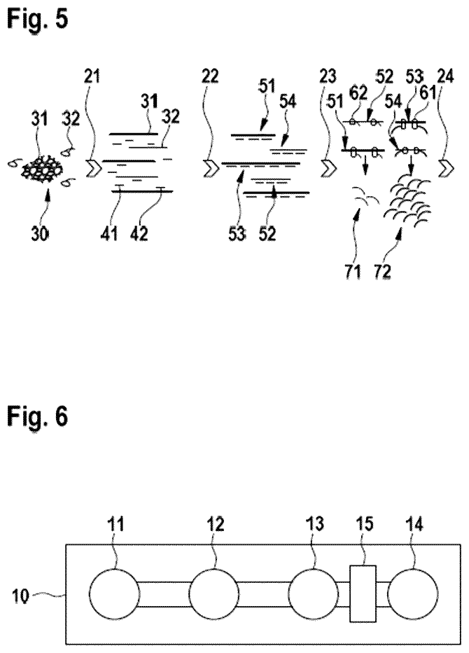

In a fourth exemplary embodiment of the invention shown in FIG. 5, the fourth exemplary embodiment is modified such that the reaction solution mixed in the second element 12 with the hybridized nucleic acids 51-54, as in the second exemplary embodiment of the method, additionally contains a reverse transcriptase 62. During the preamplification 23, the hybridized DNA strands 51, 53 are then preamplified exclusively by SD polymerase 61, while the hybridized RNA strands 52, 54 are also partially preamplified by SD polymerase 61, but predominantly following reverse transcription to cDNA, by virtue of the reverse transcriptase 62. Otherwise, the flow of the fifth exemplary embodiment of the method according to the invention corresponds to the flow of the third exemplary embodiment.

FIG. 6 shows elements of the fluidic layer of a further exemplary embodiment of the microfluidic device 10. By contrast to the device shown in FIG. 1, a fifth element 15 for the purification of the preamplification product is arranged between the third element 13 and the fourth element 14. This fifth element 15 is configured for streptavidin-based purification in order to bring the relevant biomarker loci into a particularly favorable relationship with the less relevant loci. This may increase sensitivity and specificity in the third element 13 in downstream detection methods.

When using this device, the third and fourth exemplary embodiments of the method are converted to a fifth and sixth exemplary embodiment. The modified specific sequence primers 42 have sequences that are selected so as to bind to, or next to, for example, loci relevant for viruses, bacteria, fungi, and tumors for the detection of which the microfluidic device 10 is provided. For example, these modified specific sequence primers 42 have biotin as selection markers at the 5′ end. The second amplification product 72 in these modified exemplary embodiments is specific to these sequences. After completion of the preamplification 23, and prior to the next detection step 24, an intermediate streptavidin-based purification is provided in the fifth element 15 in order to detect the associated markers in the detection step 24 with high sensitivity and specificity.

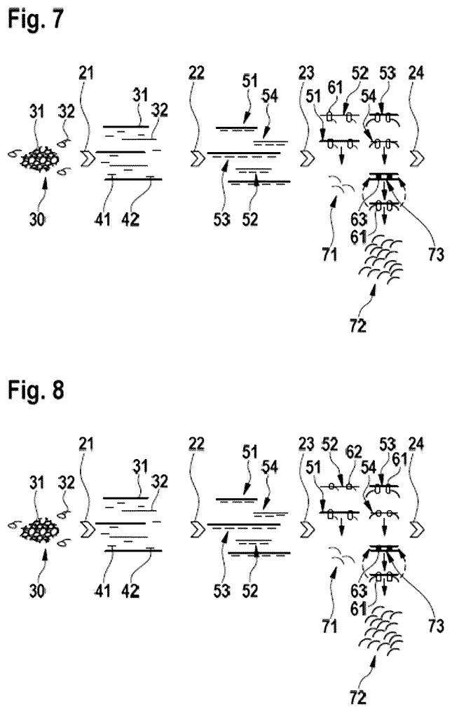

Further variations of the third and fourth exemplary embodiments into a seventh and eighth exemplary embodiments of the invention are shown in FIGS. 7 and 8. It is provided in this case that the modified specific sequence primers 42 each have a 5′ . . . GGATCNNNNN . . . 3′ sequence as a single-stranded break interface, for example. In the third element 13, Nt. AlwI is added as an endonuclease 13. Once the second preamplification product 72 is available in double-stranded form 73 after a plurality of passes of the preamplification, the endonuclease 63 binds to the single-stranded break interfaces and inserts a single-stranded break. This is detected by the SD polymerase 62, which uses it as the target point for further preamplification. If the thus obtained single strand becomes double-stranded again, then the endonuclease 63 again recognizes the single-stranded break interface on the same molecule, and the process repeats itself. Thus, the yield of the second preamplification product 72 may be further increased compared to the third and fourth exemplary embodiments.

The result of the detection reactions occurring in the fourth element 14 is ultimately output in all exemplary embodiments of the method according to the present invention by means of a user interface on the analysis device. The microfluidic device 10 can be disposed of as a disposable item after use thereof.

1. A method for preamplifying a nucleic acid mixture comprising:
denaturing the nucleic acid mixture in order to obtain single-stranded nucleic acids
hybridizing the single-stranded nucleic acids with chemically modified primers, and
isothermally preamplifying the hybridized nucleic acids by way of an SD polymerase.

2. The method according to claim 1, wherein the isothermal preamplification occurs at a temperature in the range of 40° C. to 72° C.

3. The method according to claim 2, wherein the isothermal preamplification occurs at a temperature in the range of 45° C. to 65° C.

4. The method according to claim 1, wherein the primers are ZNA primers.

5. The method according to claim 1, wherein the primers are modified random sequence primers.

6. The method according to claim 1, wherein a hybridization of the single-stranded nucleic acids further occurs with specific sequence primers

7. The method according to claim 6, wherein the specific sequence primers each comprise at least one selection marker.

8. The method according to claim 1, wherein the primers have a single-stranded break interface for an endonuclease

9. The method according to claim 1, wherein the nucleic acid mixture contains DNA and RNA.

10. The method according to claim 9, wherein the SD polymerase is an enzyme mutant of the DNA polymerase of the Bacillus stearothermophilus.

11. The method according to claim 10, wherein the SD polymerase is Bst 3.0.

12. The method according to claim 9, wherein at least one reverse transcriptase is additionally used in the isothermal preamplification.

13. A microfluidic device comprising an element for receiving a patient sample, an element for preamplifying a nucleic acid mixture in the patient sample, and an element for detecting nucleic acid biomarkers in the preamplified nucleic acid mixture, wherein the device is designed so as to carry out the preamplification by way of a method according to claim 1.
